(19) United States
(12) Patent Application Publication
(10) Pub. No.: US 2025/0033998 A1
(43) Pub. Date: Jan. 30, 2025

(54) AQUATIC INSTALLATION PREDICTIVE MAINTENANCE SYSTEM AND METHOD

(71) Applicant: PCFR SAS, Cucuron (FR)

(72) Inventors: Lane Hoy, Vaugines (FR); Xavier Darok, Verquieres (FR); Sebastien Ettling Coeffier, Cabrieres d'Aigues (FR); Rene Brunier, Vaugines (FR); Lucas Dupuis, LILLE (FR); Bruno Mouysset, EYGUIERE (FR)

(21) Appl. No.: 18/358,073

(22) Filed: Jul. 25, 2023

(51) Int. Cl. C02F 1/00; G01N 33/18; G06T 11/00

(52) U.S. Cl. CPC C02F 1/008; G01N 33/1826; G01N 33/1886; G06T 11/00; C02F 1/02; C02F 1/50; C02F 1/5209; C02F 1/66; C02F 1/686; C02F 2103/42; C02F 2209/001; C02F 2209/008; C02F 2209/02; C02F 2209/03; C02F 2209/04; C02F 2209/05; C02F 2209/06; C02F 2209/07; C02F 2209/11; C02F 2209/16; C02F 2209/18; C02F 2209/29; C02F 2209/40; C02F 2303/04

(57) ABSTRACT

The aquatic installation monitoring system (100) comprises:
at least one physical/chemical sensor (110, 117, 159, 181, 182, 183 and/or 184), configured to provide series of at least one sensed value representative of a local physical/chemical parameter, and
at least one optical sensor (115, 118), configured to provide a graphical representation of water in the aquatic installation and/or the aquatic installation, and
an aquatic installation state determination means (120), comprising a computing device configured to receive and to process at least one series of a local physical/chemical parameter and/or at least one graphical representation to determine a value representative of an aquatic installation state.

### TECHNICAL FIELD OF THE INVENTION

The present invention relates to an aquatic installation monitoring system and an aquatic installation monitoring method. It applies, in particular, to the field of water treatment and to the field of in situ water treatment. The present invention applies to recreational aquatics (pools, spas, spray pads, water features, water fountains, water parks, wellness facilities, therapy facilities, lazy rivers, etc.) and to any similar industry or market segment (evaporative cooling for energy generation and data centers, heating, ventilation and air conditioning, and fire suppression stored water management, etc.).

### BACKGROUND OF THE INVENTION

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

In current systems, the efficiency of a water treatment circuit is measured within the hydraulic circuit (in the pipes, pumps and other chemical storing tanks) which supplies a water installation.

However, such systems are inefficient in that they do not account for the shape of the installation, the circulation of water within that installation, the external parameters (such as weather forecasts), number and sizes of bathers (in the case of swimming pool), pollution in the installation (such as leaves, algae spots or stain) and the surrounding of the installation. Therefore, in order to monitor the impact of a water treatment process, operators must test the chemical state of the water in the installation in a few locations to determine whether the treatment process is successful or not and report the external factors and/or parameters. Such constraints also apply to the diagnostics to define a water treatment process to be performed and adjusted based on the external factors and/or parameters influence. Furthermore, such monitoring is also instantaneous and thus only provides a snapshot of the physical and/or chemical state of an aquatic installation. Furthermore, such monitoring is static in terms of positioning and unadaptable to increase the quality of a measure of a particular physical/chemical value correlated to the real time need of the installation taking into account any externals factors and/or parameters.

Therefore, current systems provide an inaccurate representation of the real-time and location dependent physical and/or chemical state and needs of water in an aquatic installation.

### SUMMARY OF THE INVENTION

The present invention aims at overcoming the above-mentioned drawbacks as well as other drawbacks that could be overcome although not mentioned in the description below.

The inventors have discovered that using a combination of at least one physical/chemical sensor and/or at least one optical sensor and/or at least one installation external factors and/or parameters, allows for the accurate determination of the state and the needs of an aquatic installation.

Furthermore, the inventors have discovered that using an autonomous vehicle, configured to sense at least one parameter representative of the physical and/or chemical state of a body of water in proximity to the vehicle and associate such a value with a timestamp, both the local and global physical and/or chemical states of water in an aquatic installation and/or external factors and/or parameters management may be accurately monitored.

Such a monitoring system may further comprise an optical sensor which provides alternative data points to be used to reinforce the evaluation of the state of the water in an installation or the state of the installation itself. Such an optical sensor may also provide data points to be used in combination with physical and/or chemical sensors to accurately determine the state of the water in an installation or the state of the installation itself.

Such a monitoring system may be integrated in a feedback loop associating the autonomous vehicle, external factors and/or parameters management and a hydraulic system associated with the aquatic installation.

Such a monitoring system may be associated with advanced computation capabilities, such as by using machine learning, to provide an accurate representation of the physical and/or chemical state of the water and/or diagnostics of water treatment processes to be performed on said body of water.

Such a monitoring system may ease facility management in a variety of contexts, such as aquatic facilities for example.

### BRIEF DESCRIPTION OF THE DRAWINGS

Other advantages, purposes and particular characteristics of the invention shall be apparent from the following non-exhaustive description of at least one particular system and method object of this invention, in relation to the drawings annexed hereto, in which:

FIG. 1 represents, schematically, a particular embodiment of a system object of the present invention,

FIG. 2 represents, schematically and in the form of a flowchart, a particular succession of steps of a method object of the present invention,

FIG. 3 represents, schematically, a particular embodiment of a submersible vehicle used in the system object of the present invention,

FIG. 4 represents, schematically, a particular embodiment of a floating vehicle used in the system object of the present invention,

FIG. 5 represents, schematically, a first view of a particular sensor that can be used in the system object of the present invention,

FIG. 6 represents, schematically, a second view of a particular sensor that can be used in the system object of the present invention,

FIG. 7 represents, schematically, a graph representing a succession of pH measurements at the boundary layer of a body of water for different total alkalinity values, and

FIG. 8 represents, schematically, a computer system capable of performing a method object of the present invention.

### DETAILED DESCRIPTION OF THE INVENTION

This description is not exhaustive, as each feature of one embodiment may be combined with any other feature of any other embodiment in an advantageous manner.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should be noted that the figures are not to scale.

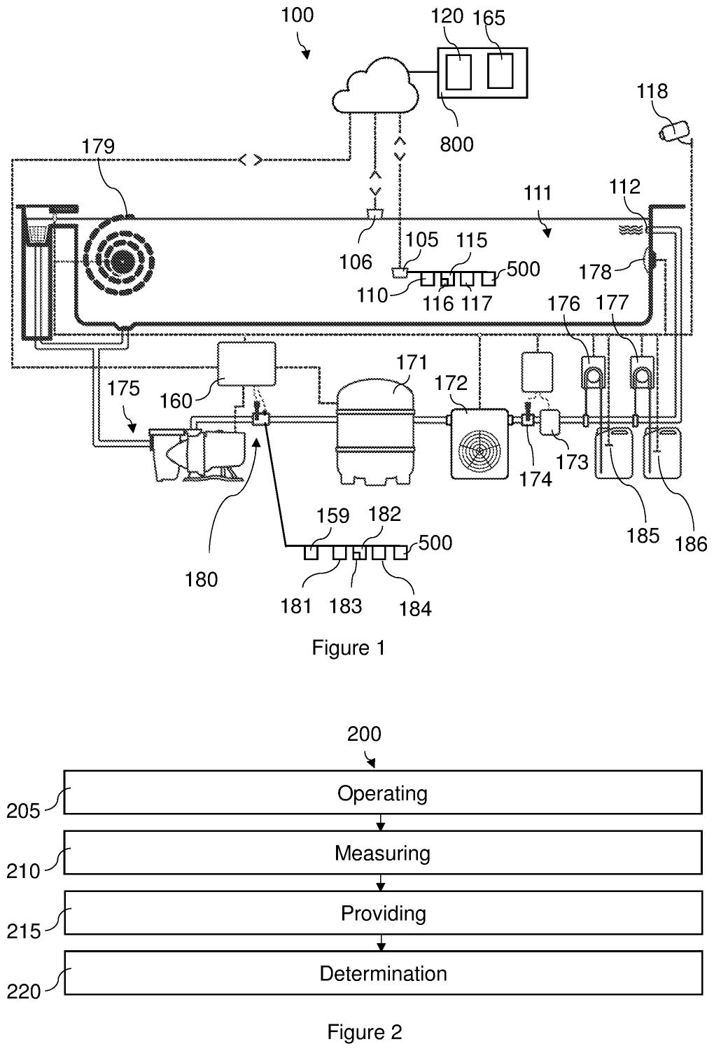

FIG. 1 represents, schematically, a particular embodiment of the system 100 object of the present invention. This aquatic installation monitoring system 100, characterized in that it comprises:
at least one physical/chemical sensor, 110, 117, 159, 181, 182, 183 and/or 184, configured to provide series of at least one sensed value representative of a local physical/chemical parameter,
at least one optical sensor, 115, 116 and/or 118, and
an aquatic installation state determination means 120, comprising a computing device configured to receive and to process at least one series of a local physical/chemical parameter and/or at least one graphical representation to determine a value representative of an aquatic installation state.

In particular embodiments, the system 100 comprises a submersible and/or floating vehicle, 105 and/or 106, comprising:
at least one said physical/chemical sensor, 110 and/or 117, configured to provide series of at least one sensed value representative of a local physical/chemical parameter, and
at least one said optical sensor, 115 and/or 116, configured to provide a graphical representation of water in the aquatic installation and/or the aquatic installation.

In particular embodiments, the system 100 comprises at least one external sensor 118, configured to provide an external graphical representation and/or video and/or picture acquisition of water in the aquatic installation and/or the aquatic installation and/or to provide external factors and/or parameters acquisition.

The submersible and/or floating vehicle, 105 and/or 106, may correspond, for example, to any manually, remotely and/or automatically dirigible vehicle adapted to the particular use case.

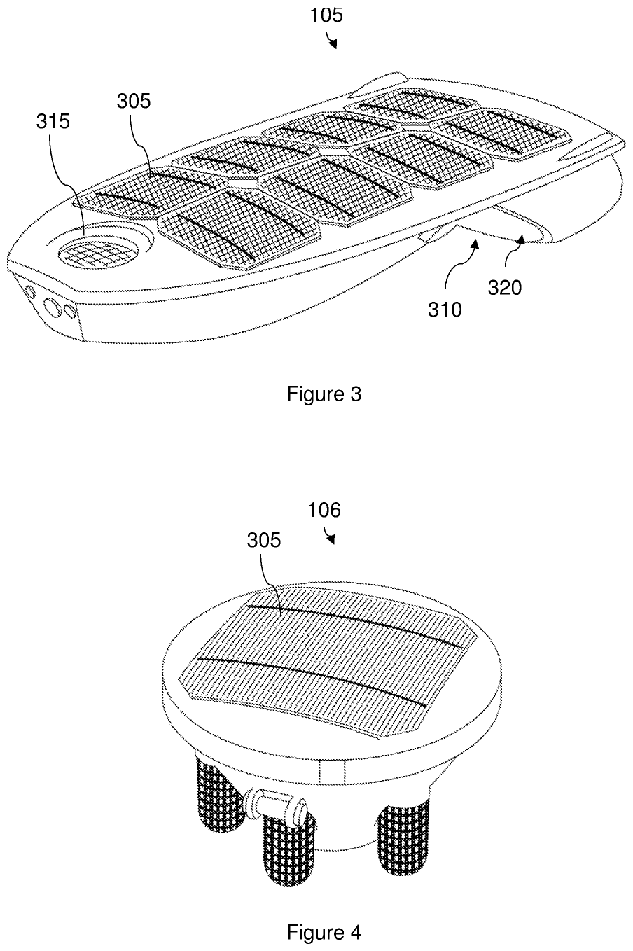

The vehicle 105 may correspond to a remotely or autonomously dirigible submarine vehicle, for example, such as shown in FIG. 3.

The vehicle 106 may also correspond to a floating pod, such as shown in FIG. 4.

In particular embodiments, such as the one shown in FIG. 1, the system 100 comprises both a submersible vehicle (ROV) 105 and a floating vehicle 106.

In particular variants, at least one submersible and/or floating vehicle, 105 and/or 106, comprises a solar panel 305 configured to power an autonomous electricity source (not represented) and/or to charge the onboarded batteries (not represented).

In particular variants, at least one submersible and/or floating vehicle, 105 and/or 106, comprises an induction current collector configured to power an autonomous electricity source (not represented).

In particular variants, at least one submersible and/or floating vehicle, 105 and/or 106, comprises a power inlet configured to be connected to a charging wire to power an autonomous electricity source (not represented).

In particular variants, at least one submersible and/or floating vehicle, 105 and/or 106, comprises a propulsion 310 system, such as an engine associated with a boat propeller. Such a propulsion 310 system allows for the vehicle, 105 and/or 106, to move around in the water in the aquatic installation 111. This propulsion system 310 may comprise rear propellers 320, configured to generate forward, backward or yaw movements, and a front propeller 315, configured to generate upward or downward movements.

Such a submersible and/or floating vehicle, 105 and/or 106, may further comprise a relative positioning coordinates acquisition means 110, configured to locate, in a three-dimensional space representative of an aquatic installation 111, the submersible vehicle, 105 and/or 106, and to provide the corresponding coordinates of the submersible and/or floating vehicle, 105 and/or 106.

Such a relative positioning coordinates acquisition means 110 is, for example, a sonar configured to provide distance values from edges of the aquatic installation 111. The distance values allow for the determination of the shape of the aquatic installation 111. Once the shape of the aquatic installation 111 is known, such distance values allow for the determination of the positioning of the submersible and/or floating vehicle, 105 and/or 106, within said aquatic installation 111.

In another variant, the relative positioning coordinates acquisition means 110 is, for example, a mechanical sensor used in coordination with a propulsion system 310 to map the shape of the aquatic installation 111 by detecting collisions of the submersible and/or floating vehicle, 105 and/or 106, with the edges of this aquatic installation 111.

In another variant, the relative positioning coordinates acquisition means 110 is, for example, detected by the external sensor 118, to map the shape of the aquatic installation 111 by detecting the edges of this aquatic installation 111 and the position of the submersible and/or floating vehicle, 105 and/or 106. In such a variant, the submersible and/or floating vehicle, 105 and/or 106 may be linked to the external sensor 118 to avoid any collision with the edge of this aquatic installation 111.

Once the shape of the aquatic installation 111 is known, information originating from original parameters of the propulsion system 310 may be used to locate the submersible and/or floating vehicle, 105 and/or 106. For example, a duration of use of the propulsion system 310, associated with a power of propulsion, may be used in a calculation to determine a distance of the submersible and/or floating vehicle, 105 and/or 106, from the last known location.

The data resulting from the physical and/or chemical sensing means 115 may further be associated to a spatial value obtained by a timestamping means configured to associate a value representative of a time of capture to the data resulting from the physical and/or chemical sensing means 115 to form a series of timestamped local physical and/or chemical value sensed.

The data resulting from the physical and/or chemical sensing means 115 may further be associated to a spatial value obtained by the relative positioning coordinates acquisition means 110 to form a series of local physical and/or chemical value sensed. This data may further be associated with environmental context values, such as pressure or time of capture for example.

The combination of the use of a timestamping means and a relative positioning coordinates acquisition means allows the formation of a series of local timestamped physical and/or chemical value sensed.

In particular variants, the submersible and/or floating vehicle, 105 and/or 106, comprises an aquatic installation local physical and/or chemical state information aggregation means.

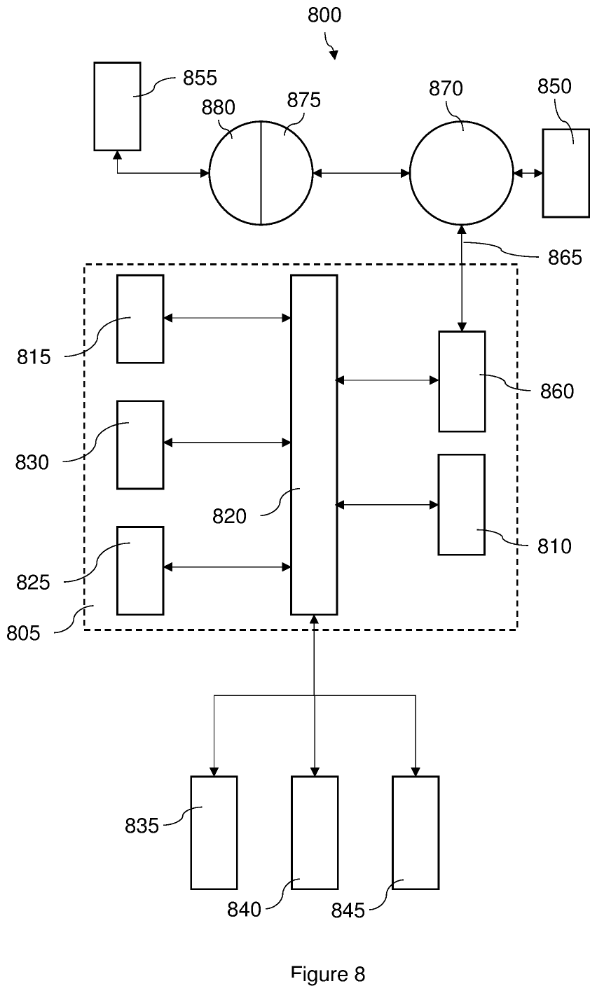

Data originating from different sensors, timestamping means or relative positioning coordinates acquisition means may be processed by an aquatic installation local physical and/or chemical state information aggregation means. Such an aggregation means is, for example, a computer software executed upon a computing device 800, such as the one shown in FIG. 8. This computing device 800 is configured to associate, in a memory, the data resulting from the relative positioning coordinates acquisition means 110, the physical and/or chemical sensor, 110 and/or 117, and/or an optical sensor, 115 and/or 116, and/or a timestamping means.

Such an association may be performed by concatenating said data in a single data stream or data frame or by creating a link between said data if such data is stored in separate database tables for example.

The timestamping means may correspond, for example, to any electronic clock used by a computing device. Such a timestamping means may be integrated into the submersible and/or floating vehicle, 105 and/or 106, or be remotely located from said submersible and/or floating vehicle, 105 and/or 106. By remotely located, it is intended that the timestamping means is linked with the submersible and/or floating vehicle, 105 and/or 106, by a communication means, such as a peer-to-peer link or a communications network link, such as the Internet for example.

The system 100 further comprises a water physical and/or chemical sensor 110. Such a water physical and/or chemical sensor 110 may be associated with a submersible and/or floating vehicle, 105 and/or 106.

Such a water physical and/or chemical sensor 110 is intended in the broadest sense, meaning that any physical and/or chemical parameter sensing device is encompassed, provided the output data of such a device is used to evaluate the physical and/or chemical state of the water in the aquatic installation 111.

The data provided by the physical/chemical sensor, 110 and/or 117, and the optical sensor, 115 and/or 116, may also be complemented by data originating from at least one further physical and/or chemical sensor, 159, 181, 182, 183 and/or 184, is situated in an analysis chamber 180, or in a pipe of a circulation system where water flows, interacting with water in at least one aquatic installation and configured to provide series of at least one sensed value representative of a physical/chemical parameter. Such a physical/chemical parameter may be representative of a state of operation of the aquatic installation and/or of the water in the aquatic installation 111.

The physical and/or chemical sensor, 110, 115, 116, 117, 159, 181, 182, 183 and/or 184, is intended in the broadest sense, meaning that any physical and/or chemical parameter sensing device is encompassed, provided the output data of such a device is used to evaluate the physical and/or chemical state of the water in an aquatic installation 111 and/or the physical and/or chemical state of the aquatic installation 111, and/or status of equipment in the aquatic installation.

Such a physical and/or chemical sensor, 110, 115, 116, 117, 159, 181, 182, 183 and/or 184, may correspond to:
a pH sensor, and/or
a total alkalinity sensor, and/or
a conductivity sensor, and/or
an oxidation-reduction potential sensor, and/or
a free chlorine sensor, and/or
a total chlorine sensor, and/or
a disinfectant rate sensor, and/or
a turbidity sensor, and/or
an optical sensor, and/or
a camera and/or video camera, and/or
an infrared sensor, and/or
an acoustic and/or sonar sensor, and/or
a temperature sensor, and/or
a flow sensor, and/or
a water movement sensor, and/or
a pressure sensor, and/or
a bacterial and/or algae activity sensor, and/or
a phosphate sensor, and/or
a nitrogen compounds sensor, and/or
a chloride sensor.

The submersible and/or floating vehicle, 105 and/or 106, further comprises an optical sensor 115. Such an optical sensor 115 corresponds, for example, to a camera or to a video camera configured to capture images of the aquatic installation 111 and/or the water in the aquatic installation 111.

In particular embodiments, such as the one represented in FIG. 1, the optical sensor 115 comprises an infrared sensor 116.

The system 100 further comprises an aquatic installation state determination means 120. Such an aquatic installation state determination means 120 corresponds to a computing device. Such a computing device is, for example, configured to execute instructions corresponding to a computer software. An example of such a computing device 800, or computer system, is shown in regard to FIG. 8.

This aquatic installation state determination means 120 may be, for example, a computer software executed upon a computing device. This aquatic installation parameter determination means 120 may use an algorithmic module or a machine learning module to link a parameter value to at least one value sensed by the submersible and/or floating vehicle, 105 and/or 106, and/or by any other sensor associated with the aquatic installation 111 and/or by any other sensor associated with an hydraulic circuit 175 associated with the aquatic installation 111.

An algorithmic module comprises a series of mathematical operations to be performed on a set or stream of data whereas a machine learning module comprises a machine learning architecture used on a training set or stream of data in order to produce a trained machine learning model that may then be used with operational data.

The aquatic installation state determination means 120 can be associated with at least one communication interface such as shown in regard to FIG. 8.

The aquatic installation state determination means 120 is configured to determine a value representative of a water and/or aquatic installation state as a function of data emitted by a physical/chemical sensor 110 and/or an optical sensor 115. The nature of the state determined is variable and dependent on the particular use case of the system 100.

In particular embodiments, such as the one represented in FIG. 1, the submersible and/or floating vehicle, 105 and/or 106, comprises a sonar sensor 117 configured to provide an output, the aquatic installation state determination means 120 being configured to determine an aquatic installation state as a function of the output provided by the sonar sensor.

Based on the sound modifications perceived by the sonar sensor, notifications and/or alerts can be emitted.

For example, a pump which starts operating provides a specific sound (vibrating signature), these vibrations follow the water flow into the aquatic installation 111. Therefore, the use of a sonar sensor allows for the measurement and analysis of these vibrations. Any change in this signature thus results from an interaction and/or a problem which has occurred between the pump and the aquatic installation 111. Such an interaction could typically correspond to a non-activation of the pump, a pump surge, a pump cavitation, an object/pollution in the pipe, a leak or a product injection. By measuring a difference between the normal state sound and the current measured sound, the diagnostic can be made. This difference can also be linked to other types of data collected.

Any sound that may occur in the aquatic installation 111 can be linked to a type, or class, of event. The determination of this class can be obtained by using a trained machine learning classifier model. Such a trained machine learning classifier model can be obtained by feeding, in a machine learning classifier device, a sample comprising sounds and the related events. Such a trained machine learning classifier model can be obtained by feeding, in a machine learning classifier device, a sample comprising the sound in installations in the absence of an event (or anomaly), the sound in installations after an event and the related events.

Such a sound can correspond to a bather diving, an object entering in the water installation, rain drops, bathers swimming or playing, air release under water by bathers, overflow problems, full skimmer baskets, different flow rate or an opening/closing of the cover.

This sound-based alert system may be used to detect a bather drowning.

In particular embodiments, the system 100 object of the present invention comprises a water physical/chemical treatment device, 172, 173, 176 and/or 177, configured to modify a water physical and/or chemical parameter as a function of the aquatic installation state determined.

Such a water physical and/or chemical treatment device may correspond to any actuator 160 suited for the particular use case of the system 100 object of the present invention. Such an actuator 160 may be one regularly used within or in combination of a hydraulic circuit or aquatic installation.

Another element may correspond to a heat pump 172 associated to an actuator (160) configured to increase or decrease the temperature of the water. Such an actuator 160 may be linked to a remote computing device 165.

Another element may correspond to a disinfectant device 173 (electrochlorinator system, ultraviolet system, or ozone generator system or any possible in situ disinfectant generator or any solid disinfectant feeder which dissolution and injection could be controlled by an actuator) associated to an actuator (160) configured to increase or decrease the activation of the disinfectant device 173. Such an actuator 160 may be linked to a remote computing device 165.

Such a disinfectant device 173 may further be associated with an analysis chamber 174 associated with at least one sensor (not referenced). Such a sensor may be configured to detect a level of bacterial and/or algae activity and/or a disinfectant adjustment need in a sample of water from the aquatic installation 111. Such a sensor may also be configured to measure free chlorine and/or total chlorine to determine combined chlorine and manage the chlorine need and adjust the electrochlorination process. Such a sensor may also be configured to detect a flow or an absence of flow in the electrochlorinator cell as a safety additional equipment to avoid any electrochlorination process with absence of flow.

Another element may correspond to a pH adjustment device 176 associated to an actuator 160 configured to increase or decrease the pH of the water. Such an actuator 160 may be linked to a remote computing device 165.

Such a pH adjustment device 176 may be associated to a pH sensor (not represented) and/or a pH adjustment chemical compound drum level 185.

Another element may correspond to a disinfectant (such as liquid chlorine or sodium hypochlorite or any liquid disinfectant) release device 177 associated to an actuator 160 configured to increase or decrease the concentration of disinfectant in the water. Such an actuator 160 may be linked to a remote computing device 165.

Such a disinfectant release device 177 may be associated to a disinfectant chemical compound drum level 186.

Such a disinfectant release device 177 may further be associated with an analysis chamber 180 associated with at least one sensor (159, 181, 182, 183 and/or 184). Such a sensor may be configured to detect a level of bacterial activity and/or a disinfectant adjustment need in a sample of water from the aquatic installation 111. Such a sensor may also be configured to measure the disinfectant rate and/or the free chlorine and/or total chlorine to determine combined chlorine and/or the disinfectant residuals and manage the chlorine and/or disinfectant need and adjust the disinfectant release process. Such a sensor may also be configured to detect a flow or an absence of flow in the pipe and/or in the analysis chamber as a safety additional equipment to avoid any disinfectant release process with absence of flow.

Another element may correspond to an algicide rate adjustment device (not referenced) associated to an actuator 160 configured to increase the algicide rate in the water base. Such an actuator 160 may be linked to a remote computing device 165.

Such an algicide rate adjustment device (not referenced) may be associated to a bacterium and/or algae activity sensor (located in an analysis chamber 180) or an algicide rate adjustment chemical compound drum level. Such a sensor may be configured to detect a level of bacterial activity in a sample of water from the installation 111.

Another element may correspond to a flocculant and/or clarifier adjustment device (not referenced) associated to an actuator 160 configured to decrease the turbidity in the water base. Such an actuator 160 may be linked to a remote computing device 165.

Such a flocculant and/or clarifier adjustment device (not referenced) may be associated to a turbidity sensor (located in an analysis chamber 180) or a flocculant and/or clarifier adjustment chemical compound drum level. Such a sensor may be configured to detect a level of turbidity in a sample of water from the installation 111.

Another element may correspond to a liquid additive treatment adjustment device (not referenced) associated to an actuator 160 configured to improve the water treatment. Such an actuator 160 may be linked to a remote computing device 165.

Such a liquid additive treatment adjustment device (not referenced) may be associated to a specific sensor (located in an analysis chamber 180) or a liquid additive drum level (not referenced). Such a sensor may be configured to detect the need for an additive treatment injection in a sample of water from the installation 111.

Another element may correspond to a light 178 configured to illuminate the water in the aquatic installation 111 and associated to an actuator (not referenced) configured to activate or deactivate the light 178. Such an actuator 160 may be linked to a remote computing device 165. The activation of such a light 178 increases the performance of image-based sensors, such as particle sensors and/or turbidity/clarity sensors.

Another element may correspond to an installation cover 179 configured to selectively cover the aquatic installation 111.

This installation cover 179 may be associated with an actuator 160 configured to open or close the cover 179, for purposes of safety, water pollution reduction, evaporation control, and/or energy cost reduction for example. Such an actuator 160 may be linked to a remote computing device 165.

This installation cover 179 may be associated with a sensor (not referenced) and/or with an external sensor 118 configured to monitor the position of the cover 179.

In particular embodiments, the system 100 object of the present invention comprises a command emitter configured to emit a command representative of a target operational value for an actuator 160 interacting with the physical and/or chemical state of the aquatic installation.

The command emitter is, for example, a computer software executed upon a computing device and associated to a communication means linking the emitter to the actuator 160.

This command emitter is, for example, activated as a function of the result of a comparison between the value of a parameter sensed by the submersible and/or floating vehicle, 105 and/or 106, and a predetermined target value. Such a target value corresponds to, for example, a value representative of a desired physical and/or chemical state of the water in the aquatic installation 111.

Such a command may correspond to, but is not limited to, for example:
a release or the end of a release of a chemical compound in the water of the aquatic installation 111, and/or
an increase or a decrease in a water pump activation, and/or
an increase or a decrease in a water pump flow, and/or
an increase or a decrease in a water heater activation.

In particular embodiments, the water physical/chemical sensor 110 and/or the external sensor 118 is configured to measure a flow intensity from an inlet 112 in the installation, the aquatic installation state determination means 120 being configured to determine a pump flow efficiency as a function of the flow intensity.

The higher the flow is, the greater the travelled distance by the water pushed out of the inlet is. This also correlates with a higher water movement at the surface.

In particular embodiments, the water movement at the surface is measured by an external sensor 118 and correlated to the water flow through the actuator 160.

Therefore, the measurement of the intensity of such a flow allows the determination of an anomaly in a hydraulic circuit associated with the inlet 112. Such an intensity may be measured in an initial state of the hydraulic circuit, corresponding to a nominal operating mode, by positioning the submersible and/or floating vehicle, 105 and/or 106, at a particular distance of the inlet, and/or by the distance of the water movement from the inlet measured by an external sensor 118. Then, this intensity can be measured in the same location regularly, an anomaly being detected when the intensity measured is substantially different from the nominal intensity.

Based on the loss of the flow intensity out of the inlet, the state determination means 120 can detect the loss of pump flow efficacy and diagnose the cause in correlation with the filter pressure and pump speed.

Below, several examples of diagnosis are provided:
Example 1: for the same filter pressure and pump speed, a loss of flow intensity can be generated by a leak in the pipes after the filter 171.
Example 2: for the same pump speed, an increase of pressure in the filter 171 and a loss of flow intensity are linked to a saturated filter 171 and generate an alert and/or can trigger the activation of a filter cleaning.
Example 3: a higher pump speed increases the flow intensity. If not, the state determination means 120 detects if it is a leak (same filter pressure) or a saturated filter (increase of the filter pressure).

In particular embodiments, the aquatic installation state determination means 120 is configured to determine a water clarity value as a function of a graphical representation provided.

Such a water clarity value may be determined as a function of the distance required for the optical sensor of the submersible and/or floating vehicle, 105 and/or 106, to detect a determined or predetermined target. Such a target may correspond, for example, to a water inlet of an aquatic installation 111. The definition of such a target may be initiated by an operator, by positioning the submersible and/or floating vehicle, 105 and/or 106, in a particular location of the aquatic installation 111, commanding the capture of an image by the optical sensor and storing the coordinates of the vehicle at the moment of capture. These coordinates allow later image captures in the same coordinates, closer to the target or farther from the target.

Below, several examples of diagnosis are provided:
Example 1: low point acquisition (turbid water) and a high pressure in the filter 171 of a hydraulic circuit associated with the aquatic installation 111 may trigger a filter cleaning command and/or emission of an alert.
Example 2: low point acquisition (turbid water) and an adequate filter pressure can trigger the increase of the pump speed (or filtration time) if chemical state values are not good enough in the aquatic installation 111.

In particular embodiments, the aquatic installation state determination means 120 is configured to determine a value representative of the presence of particles in the water as a function of a graphical representation provided.

Particles may be detected, for example, during the night, by switching on lights 178 aiming at the aquatic installation 111 and capturing images of the reflection of particles inside the water in the installation using an external sensor 118 and/or an optical sensor 115. These reflections can be counted to give an average of the particles inside the water.

Based on this value, a pump speed and filtration time can be adjusted to allow the deposition of those particles inside a filter 171 (the lowest the speed is, the easiest the particles are kept in the filter) and/or the use of a coagulant and/or flocculant product can be piloted to help the lower the number of particles inside the filter 171.

In particular embodiments, the aquatic installation state determination means 120 is configured to determine a value representative of the presence of the nature of an impurity in the water as a function of a graphical representation provided using an external sensor 118 and/or an optical sensor 115.

Such an impurity may be detected via an image processing algorithm.

When such an impurity is detected, as a new color or form detection on a coating of the aquatic installation 111, an algorithm may determine the nature of the spot based on its form and color.

Below, examples of such detections are provided:
Example 1: In the case of leaves, dark spots which move with the flow can be detected, and an aquatic installation 111 cleaner can be activated to remove said leaves.
Example 2: In the case of algae spot detection (green spots), an aquatic installation 111 cleaner and a disinfection boost correlated with a filtration time and pump speed increase are activated, and/or an aquatic installation 111 cleaner can be activated to brush said algae.
Example 3: In the case of mushrooms detection (pink spots), or rust (brown spot) an alert can be provided, this alert further provided instructions detailing how to remove said mushrooms or rust.

In particular embodiments, the aquatic installation state determination means 120 is configured to determine a value representative of the presence of an animal in the water as a function of a graphical representation provided.

Such a presence may be detected by an image processing algorithm configured to recognize shapes of animals or humans, for example. In other variants, the amount of noise generated by turbulences in the water associated with the motions of the animal or human may trigger the detection of this presence.

In particular embodiments, the aquatic installation state determination means 120 is configured to determine a value representative of a movement pattern of the animal in the water as a function of a graphical representation provided.

Such a presence may be detected by an image processing algorithm configured to recognize types or speed of movements of animals or humans, for example. In other variants, the amount of noise generated by turbulences in the water associated with the types of movements of the animal or human may trigger the detection of this presence.

It is possible to secure access to an aquatic installation 111 by registering authorized persons in an area around the pool. Such persons may be associated with a picture, for example, and recognized by the optical sensor 115 and/or 118.

Moreover, the detection of a human size detection further allows the prevention of children drowning by emitting an alert in case of children presence close to the aquatic installation 111.

By analyzing the body movement, it is possible to differentiate a bather swimming from a bather drowning.

This system can also measure the number of bathers at the same time in the aquatic installation 111, in order to adjust the water treatment consequently, based on the number of bathers, their size and activities, to neutralize the caused pollution by disinfectant injection and pH adjustment.

In particular embodiments, the aquatic installation state determination means 120 is configured to determine the temperature of the water based upon the output of an infrared sensor 116 of the optical sensor 115 and/or 118.

The use of such an infrared capture provides the temperature flow in the water and at its surface. Correlated to an algorithm, the temperature value of the water can be determined.

Such an information may be used in the following scenarios:
Example 1: a non-homogenous temperature detected at the pool surface can trigger a command to the pump to increase its speed or its filtration time to favorize the uniformity of the temperature.
Example 2: an optimum filtration time and pump speed, and a non-homogeneous temperature detected, can trigger a command to increase the heat pump working time and/or will close the cover if any.
Example 3: the infrared capture can determine the evaporation of water in real time and close the cover (if any) to prevent water, total alkalinity and temperatures losses by evaporation, and/or reduce the heat pump work to lower the water temperature to reduce these losses.

In particular embodiments, the system 100 further comprises a remote computing device configured to control, register and adjust a set of predetermined aquatic installation 111 operational parameters values (such as the pH or total alkalinity for example).

The remote computing device may be, for example, operated as such:
All the data is routed by a traffic manager to a dedicated cloud (computing device and/or computer software):
   in each computing device and/or computing software, an algorithm manages the data's individual value from the camera acquisition of at least one vehicle, 105 and/or 106, and/or acquired by the external sensor 118, and
   in a second step, an algorithm manages a combined global data value, and sends this data to a big data management module using the traffic manager.
The global data from at least one vehicle, 105 and/or 106, and an external sensor 118 are compared to a user-set point:
   in case of differences between these values, the aquatic installation 111 operational parameters are adjusted using water treatment and equipment piloting improvements,
   in parallel, a timestamped three-dimensional mapping of the aquatic installation 111 is recorded, and risk-determining algorithms are executed to determine the presence and severity of risks in the water aquatic installation 111 a plurality of zones, and
   a timestamped three-dimensional map is generated comprising a color code for the pool risks to identify the risk prone zones in a simplified manner.

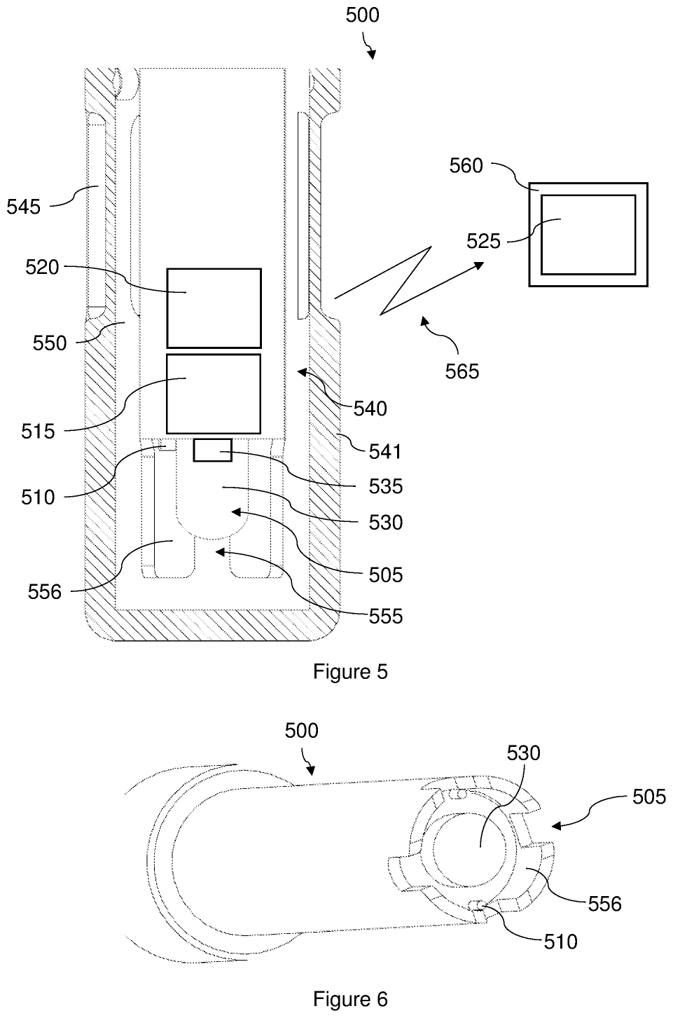

In particular embodiments, the system 100 comprises a total alkalinity measurement device, such as shown in FIGS. 5 and 6, comprising:
a pH probe 505 configured to measure pH at the boundary layer of a body of water, corresponding to the water physical and/or chemical sensor 110 of FIG. 1,
a floating reference device 510 in proximity of the pH probe,
a probe controller 515, configured to sequentially activate and deactivate the pH probe,
a pH measurement variation detection device 520, configured to detect a variation of pH measurement in a sequence of pH probe measurements, and
an aquatic total alkalinity value determination device 525, configured to determine an aquatic total alkalinity value of the body of water as a function of the pH measurement variation detected.

The pH probe 505 can be of any type known to a person skilled in the art that is suited for the particular implementation and intended use of the system 500. Such a pH probe 505 may differ in nature depending on the context of use of the system 500. For example, in a swimming pool, the pH probe 505 may comprise an oxidation-reduction potential sensor 535.

The objective of the pH probe 505 is to allow for the reproductible measurement of the pH in a body of water. Such a pH probe 505 is typically electronic and requires the supply of electrical energy to function. Such a pH probe 505 may further comprise a digital switch, allowing for the selective activation/deactivation of at least part of the core components of the pH probe 505.

The pH probe 505 may be mechanically located at the distal end of a sensor body, such as shown in FIGS. 5 and 6. The purpose of such a sensor body is the insertion in the body of water and, in preferred embodiments, within an analysis chamber 540.

The reference floating device 510, sometimes called “solution earth” or “liquid junction”, can correspond to any electrically conductive electrode or pin configured to normalize the signal sensed by the pH probe 505, avoiding electric noise in the proximity of the pH probe 505.

In the example shown in FIGS. 5 and 6, the reference floating device 510 comprises two electrodes, each located on a different side of a sensor 535 of the probe 505. Such electrodes may be diametrically opposed, with the sensor 535 acting as the center of a circle in which both electrodes are located on the periphery of said circle, for example. Such electrodes and the sensor 535 may be geometrically aligned.

In particular embodiments, such as the one shown in FIG. 5, the pH probe 505 comprises:
the floating reference device 510,
a microporous glass bulb membrane 530, and
an oxidation-reduction potential sensor 535.

pH measurement is based on the relationship between the concentration of H+ ions in tested water and the difference in electrochemical potential which is established in the lead-free glass bulb membrane of the probe. This lead-free bulb is specifically designed to be selective to H+ ions concentration.

In general, the pH probe 505 is made of a simple electronic amplifier and a combined electrode, which consists of two electrodes: one whose potential is known and constant and the other whose potential varies with the pH.

Once the probe 505 is in contact with water, the H+ ions exchange on the glass bulb, creating an electrochemical potential across the bulb. The electronic amplifier detects the difference in electrical potential between the two electrodes generated in the measurement and converts the potential difference to pH units.

The pH value is determined by correlation because the potential difference between the two electrodes evolves proportionally to the pH according to the Nernst equation.

The probe controller 515 is, for example, an electronic circuit configured to electrically or electronically activate and deactivate, or connect and disconnect, the pH probe 505 or the sensor of said pH probe 505. Such an activation/deactivation or connection/disconnection may be performed by cutting and restoring power supply to the pH probe 505 or sensor or by emitting an activation/deactivation or connection/disconnection command to said pH probe 505 or sensor or relay.

The terms “activate and deactivate” relate to any hardware or software level activation/deactivation and/or to the connection/disconnection of the pH probe 505.

The probe controller 515 may itself be activated as a function of a command emitted by a computing device, located on site and mechanically connected to the pH probe 505 and/or the probe controller 515 or remotely located and connected to the probe controller 515 by way of a data connection.

The probe controller 515 may comprise, for example, a computer software executed upon a computing device, said computer software triggering the activation/deactivation or connection/disconnection of the pH probe 505. Such a computer software may correspond, for example, to a particular firmware or driver. Such a computer software may be updated remotely, and such an update may be automatically installed in the system 500.

The probe controller 515 may be configured to activate or connect the pH probe 505 periodically. The pH probe may be activated or physically connected, for example, every 60 seconds. Such an activation or physical connection may be conditional, for example to the activation of a water displacement pump. The rate of measurement may be variable depending on the mode configured. The duration of measurement may depend on the water's stability, so the pH measurement last until the measured pH is sufficiently stable.

Such an activation/deactivation can be performed by an electronic relay.

The pH measurement variation detection device 520 is, for example, an electronic device associated with the pH probe 505, configured to record a succession of pH values measured by the pH probe 505 and to compute, from said succession, a measurement variation value. Such a measurement variation value may be computed by the subtraction of a recent value from an older value.

The measured variation may be performed on immediately subsequent measured pH values or be sampled according to a particular sampling rule. Such variation may also be performed on an aggregate values of measured pH values.

For example, the pH measurement variation detection device 520 may be configured to subtract the average measured pH value during a specific, more recent timeframe from the average measured pH value during a specific, older timeframe.

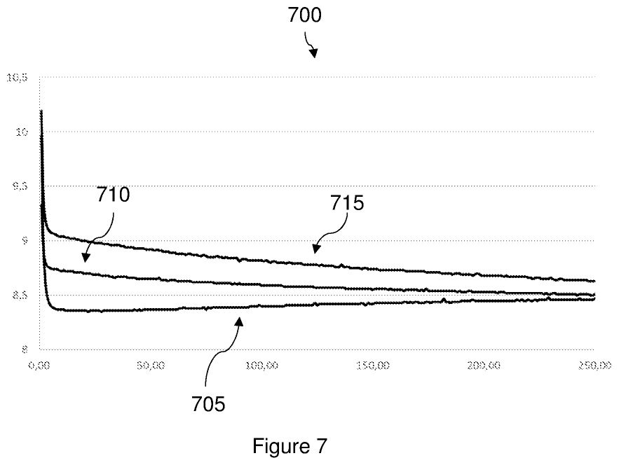

For example, the pH measurement variation detection device 520 may be configured to compute determine a mathematical function fitting a succession of data points linking measured pH to time of measurement since an initial measurement. Such an example is shown in FIG. 7. In other examples, the pH measurement variation detection device 520 may be configured to store, in a memory, a succession of data points linking measured pH to time of measurement since an initial measurement.

The system 500 may further comprise a timestamping means, configured to associate a time of measurement to a sensed pH value by the pH probe 505.

The action of repeatedly measuring the pH in the same water sample induces variations in the measurement of pH, the magnitude of these variations being dependent on the total alkalinity of the water. Such a pH measurement variation detection device 520 may also correspond to a computer software executed upon a computing device.

The pH measurement variation detection device 520 may operate remotely from the pH probe 505. In such a case, the system 500 may further comprise a communication means 565 to transmit data from the pH probe 505 to the pH measurement variation detection device 520. In such a case, the pH measurement variation detection device 520 may correspond to a computer program executed by a computing server, accessible on the cloud, via a data network such as the Internet for example.

The aquatic total alkalinity value determination device 525 is, for example, an electronic device associated with the pH measurement variation detection device 520, configured to associate a total alkalinity value to the measured variation.

For example, the total alkalinity value determination device 525 may be configured to compute the derivative of a mathematical function fitting a succession of data points linking measured pH to time of measurement since an initial measurement. Such an example is shown in FIG. 7.

The total alkalinity value determination device 525 may be configured to associate, with specific or ranges of said derivatives, a specific or a range of total alkalinity value.

For example, in FIG. 7:
a first series 705 of pH measurements (Y-axis), at specific times (X-axis), measured in minutes, since an initial measurement, for a total alkalinity value of 220 mg/l,
a second series 710 of pH measurements (Y-axis), at specific times (X-axis), measured in minutes, since an initial measurement, for a total alkalinity value of 125 mg/l, and
a third series 715 of pH measurements (Y-axis), at specific times (X-axis), measured in minutes, since an initial measurement, for a total alkalinity value of 19 mg/l.

Obtaining such series linking pH to alkalinity value relationship can be performed by empirically measuring, for different values of total alkalinity and a determined activation/connection frequency for the pH sensor, values of pH in the boundary layer of a body of water and storing these series in a memory. The number of such tests to be performed is limited in terms of scope, considering the limited number of values for alkalinity.

Such a total alkalinity value may be a mathematical function of the measured variation. Such a mathematical function may be performed by determining a regression function based upon the pH series captured, or derivative values of these series, as well as the operational parameters associated with the capture.

Such derivative values may be, for example, any type of averages or parameters of derivative functions.

For example, the following mathematical formula may be used:

$$A l k = - 0.0001 ( A V {G}_{1} - A V {G}_{2} ) + 0.1468$$

Where:
Alk designates the total alkalinity value,
AVG₁ designates the average pH values measured from 20 seconds to 80 seconds after the initial measurement,
AVG₂ designates the average pH values measured from 300 seconds to 360 seconds after the initial measurement, and
Such a function may be approximated to Alk=(AVG₁-AVG₂).

From such a function, the following correspondence table may be obtained:

| Total alkalinity value | AVG₁ − AVG₂ |
|---|---|
| 10 | 0.1368 |
| 20 | 0.1268 |
| 30 | 0.1168 |
| 40 | 0.1068 |
| 50 | 0.0968 |
| 60 | 0.0868 |
| 70 | 0.0768 |
| 80 | 0.0668 |
| 90 | 0.0568 |
| 100 | 0.0468 |
| 110 | 0.0368 |
| 120 | 0.0268 |
| 130 | 0.0168 |
| 140 | 0.0068 |
| 150 | −0.0032 |
| 160 | −0.0132 |
| 170 | −0.0232 |
| 180 | −0.0332 |
| 190 | −0.0432 |
| 200 | −0.0532 |

Such a total alkalinity value may be determined as a function of the measured variation and a preset threshold value, representative of a particular total alkalinity value.

Such an aquatic total alkalinity value determination device 525 may also correspond to a computer software executed upon a computing device.

The aquatic total alkalinity value determination device 525 may operate remotely from the pH probe 505 and/or the pH measurement variation detection device 520. In such a case, the system 500 may further comprise a communication means 565 to transmit data from the pH measurement variation detection device 520 to the aquatic total alkalinity value determination device 525. In such a case, the aquatic total alkalinity value determination device 525 may correspond to a computer program executed by a computing server, accessible on the cloud, via a data network such as the Internet for example.

In particular embodiments, the pH probe controller 515 is configured to sequentially activate and deactivate, or connect and disconnect, the pH probe 505 in a body of water with no flow. Such a state may be reached by stopping a pumping system introducing water in the body of water. In particular variants, the pH probe 505 may be activated after an absence of flow is detected (by a flow sensor, for example). In particular variants, a chamber in which the pH probe 505 is located may comprise valves that may be closed prior to the operation of the pH probe 505 activation/deactivation or connection/disconnection sequence.

The terms “body of water with no flow” designate a body of water with limited water flowing. In such a body of water, the water may circulate, but limited new water may enter.

In particular embodiments, the pH probe 505 is configured to be positioned in a small-volume body of water. Such a small volume may correspond to, for example, 1 to 2 milliliters.

The terms “small-volume body of water” designate a body of water in which the chemical reaction taking place during an interval of deactivation/activation, or connection/disconnection, of the pH probe 505 provides significant impact on the pH measure so as to show a variation between two successive measurements of the pH by the pH probe 505.

In particular embodiments, the system 500 object of the present invention comprises an analysis chamber 540, comprising an opening 545, a main volume 550 connected to the opening 545 and a recess 555 in the main volume 550, the pH probe 505 being in contact with the water in the recess 555.

The analysis chamber 540 may comprise a sensor housing 541 delimiting an internal volume in which the pH probe 505 or a sensor body associated with said pH probe 505 may be inserted.

The analysis chamber 540 is preferably configured to limit the flow of water and the volume of water in proximity to the pH probe 505. Such a configuration may be performed by selecting dimensions that limit the quantity of water entering the analysis chamber 540.

The analysis chamber 540 comprises an opening 545, of arbitrary dimensions, which allows for the passage of water from the body of water to the proximity of the pH probe 505.

The analysis chamber 540 comprises a main volume 550, defined for example by the interior dimensions of the sensor housing 541.

The analysis chamber 540 comprises a recess 555, defined by a subset of the interior dimensions of the sensor housing 541. In particular embodiments, the recess 555 is formed by crenellated sensor body extensions 556 associated to the pH probe 505, said crenellated sensor body extensions 556 limiting the movement of water in the proximity of the pH probe 505.

There are many possible configurations of the analysis chamber 540. Such configurations preferably limit the quantity of water in proximity to the pH probe 505 and/or limit the movement of water in proximity of the pH probe 505.

In particular embodiments, the system 500 object of the present invention comprises a remote computing device 560 comprising the aquatic total alkalinity value determination device 525 and a communication means 565 between the pH measurement variation detection device 520 and the aquatic total alkalinity value determination device 525.

Such a remote computing device 560 may correspond to, for example, a computing server hosted remotely and accessible through a data network, such as the Internet for example.

In particular embodiments, the aquatic total alkalinity value determination device 525 operates an algorithm and/or a trained machine learning model to associate an aquatic total alkalinity value with a variation in measured pH.

In particular embodiments, the pH probe 505 is configured to measure the pH of the body of water in a swimming pool.

In particular embodiments, the pH probe 505 is configured to measure the pH of the body of water in a pipe.

FIG. 2 represents, schematically, a particular succession of steps of the method 200 object of the present invention. This aquatic installation monitoring method 200 comprises:
a step 205 of operating:
   at least one water physical/chemical sensor, and
   at least one optical sensor.
   a step 210 of measuring a local physical/chemical state of the water environment in the proximity of a submersible and/or floating vehicle and/or of measuring at least one external factor and/or parameter using the sensor 118,
   a step 215 of providing a sensed graphical representation of the water and/or aquatic installation, and
   an aquatic installation state determination step 220, comprising a computing device configured to receive and to process measures of a local physical/chemical state of the water and/or graphical representations of the water to determine a value representative of an aquatic installation state and/or of its environment.

Particular implementations of the method 200 object of the present invention are disclosed in relation to the system 100 object of the present invention.

FIG. 8 represents a block diagram that illustrates an example computer system 800 with which an embodiment may be implemented. In the example of FIG. 8, a computer system 805 and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

The computer system 805 includes an input/output (IO) subsystem 820 which may include a bus and/or other communication mechanism(s) for communicating information and/or instructions between the components of the computer system 805 over electronic signal paths. The I/O subsystem 820 may include an I/O controller, a memory controller and at least one I/O port. The electronic signal paths are represented schematically in the drawings, for example as lines, unidirectional arrows, or bidirectional arrows.

At least one hardware processor 810 is coupled to the I/O subsystem 820 for processing information and instructions. Hardware processor 810 may include, for example, a general-purpose microprocessor or microcontroller and/or a special-purpose microprocessor such as an embedded system or a graphics processing unit (GPU) or a digital signal processor or ARM processor. Processor 810 may comprise an integrated arithmetic logic unit (ALU) or may be coupled to a separate ALU.

Computer system 805 includes one or more units of memory 825, such as a main memory, which is coupled to I/O subsystem 820 for electronically digitally storing data and instructions to be executed by processor 810. Memory 825 may include volatile memory such as various forms of random-access memory (RAM) or other dynamic storage device. Memory 825 also may be used for storing temporary variables or other intermediate information during execution of instructions to be executed by processor 810. Such instructions, when stored in non-transitory computer-readable storage media accessible to processor 810, can render computer system 805 into a special-purpose machine that is customized to perform the operations specified in the instructions.

Computer system 805 further includes non-volatile memory such as read only memory (ROM) 830 or other static storage device coupled to the I/O subsystem 820 for storing information and instructions for processor 810. The ROM 830 may include various forms of programmable ROM (PROM) such as erasable PROM (EPROM) or electrically erasable PROM (EEPROM). A unit of persistent storage 815 may include various forms of non-volatile RAM (NVRAM), such as FLASH memory, or solid-state storage, magnetic disk, or optical disk such as CD-ROM or DVD-ROM and may be coupled to I/O subsystem 820 for storing information and instructions. Storage 815 is an example of a non-transitory computer-readable medium that may be used to store instructions and data which when executed by the processor 810 cause performing computer-implemented methods to execute the techniques herein.

The instructions in memory 825, ROM 930 or storage 815 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

Computer system 805 may be coupled via I/O subsystem 820 to at least one output device 835. In one embodiment, output device 835 is a digital computer display or Human Machine Interface. Examples of a display that may be used in various embodiments include a touch screen display or a light-emitting diode (LED) display or a liquid crystal display (LCD) or an e-paper display. Computer system 805 may include other type(s) of output devices 835, alternatively or in addition to a display device. Examples of other output devices 835 include printers, ticket printers, plotters, projectors, sound cards or video cards, speakers, buzzers or piezoelectric devices or other audible devices, lamps or LED or LCD indicators, haptic devices, actuators, or servos.

At least one input device 840 is coupled to I/O subsystem 820 for communicating signals, data, command selections or gestures to processor 810. Examples of input devices 840 include touch screens, microphones, still and video digital cameras, alphanumeric and other keys, keypads, keyboards, graphics tablets, image scanners, joysticks, clocks, switches, buttons, dials, slides.

Another type of input device is a control device 845, which may perform cursor control or other automated control functions such as navigation in a graphical interface on a display screen, alternatively or in addition to input functions. Control device 845 may be a touchpad, a mouse, a trackball, or cursor direction keys for communicating direction information and command selections to processor 810 and for controlling cursor movement on display 835. The input device may have at least two degrees of freedom in two axes, a first axis (e.g., x) and a second axis (e.g., y), that allows the device to specify positions in a plane. Another type of input device is a wired, wireless, or optical control device such as a joystick, wand, console, steering wheel, pedal, gearshift mechanism or other type of control device. An input device 840 may include a combination of multiple different input devices, such as a video camera and a depth sensor.

In another embodiment, computer system 805 may comprise an internet of things (IoT) device in which one or more of the output device 835, input device 840, and control device 845 are omitted. Or, in such an embodiment, the input device 840 may comprise one or more cameras, motion detectors, thermometers, microphones, seismic detectors, other sensors or detectors, measurement devices or encoders and the output device 835 may comprise a special-purpose display such as a single-line LED or LCD display, one or more indicators, a display panel, a meter, a valve, a solenoid, an actuator or a servo.

Computer system 805 may implement the techniques described herein using customized hard-wired logic, at least one ASIC or FPGA, firmware and/or program instructions or logic which when loaded and used or executed in combination with the computer system causes or programs the computer system to operate as a special-purpose machine. According to one embodiment, the techniques herein are performed by computer system 805 in response to processor 810 executing at least one sequence of at least one instruction contained in main memory 825. Such instructions may be read into main memory 825 from another storage medium, such as storage 815. Execution of the sequences of instructions contained in main memory 825 causes processor 810 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage 815. Volatile media includes dynamic memory, such as memory 825. Common forms of storage media include, for example, a hard disk, solid state drive, flash drive, magnetic data storage medium, any optical or physical data storage medium, memory chip, or the like.

Storage media is distinct from but may be used in conjunction with transmission media. Transmission media participates in transferring information between storage media. For example, transmission media includes coaxial cables, copper wire and fiber optics, including the wires that comprise a bus of I/O subsystem 820. Transmission media can also take the form of acoustic or light waves, such as those generated during radio-wave and infra-red data communications.

Various forms of media may be involved in carrying at least one sequence of at least one instruction to processor 810 for execution. For example, the instructions may initially be carried on a magnetic disk or solid-state drive of a remote computer. The remote computer can load the instructions into its dynamic memory and send the instructions over a communication link such as a fiber optic or coaxial cable or telephone line using a modem. A modem or router local to computer system 805 can receive the data on the communication link and convert the data to a format that can be read by computer system 805. For instance, a receiver such as a radio frequency antenna or an infrared detector can receive the data carried in a wireless or optical signal and appropriate circuitry can provide the data to I/O subsystem 820 such as place the data on a bus. I/O subsystem 920 carries the data to memory 825, from which processor 810 retrieves and executes the instructions. The instructions received by memory 825 may optionally be stored on storage 815 either before or after execution by processor 810.

Computer system 805 also includes a communication interface 860 coupled to bus 820. Communication interface 860 provides a two-way data communication coupling to network link(s) 865 that are directly or indirectly connected to at least one communication network, such as a network 870 or a public or private cloud on the Internet. For example, communication interface 860 may be an Ethernet networking interface, integrated-services digital network (ISDN) card, cable modem, satellite modem, or a modem to provide a data communication connection to a corresponding type of communications line, for example an Ethernet cable or a metal cable of any kind or a fiber-optic line or a telephone line. Network 870 broadly represents a local area network (LAN), wide-area network (WAN), campus network, internetwork, or any combination thereof. Communication interface 860 may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, communication interface 860 sends and receives electrical, electromagnetic, or optical signals over signal paths that carry digital data streams representing various types of information.

Network link 865 typically provides electrical, electromagnetic, or optical data communication directly or through at least one network to other data devices, using, for example, satellite, cellular, Wi-Fi, or BLUETOOTH technology. For example, network link 865 may provide a connection through a network 870 to a host computer 850.

Furthermore, network link 865 may provide a connection through network 870 or to other computing devices via internetworking devices and/or computers that are operated by an Internet Service Provider (ISP) 875. ISP 875 provides data communication services through a world-wide packet data communication network represented as internet 880. A server computer 855 may be coupled to internet 880. Server 855 broadly represents any computer, data center, virtual machine, or virtual computing instance with or without a hypervisor, or computer executing a containerized program system such as DOCKER or KUBERNETES. Server 855 may represent an electronic digital service that is implemented using more than one computer or instance and that is accessed and used by transmitting web services requests, uniform resource locator (URL) strings with parameters in HTTP payloads, API calls, app services calls, or other service calls. Computer system 805 and server 855 may form elements of a distributed computing system that includes other computers, a processing cluster, server farm or other organization of computers that cooperate to perform tasks or execute applications or services. Server 855 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server 855 may comprise a web application server that hosts a presentation layer, application layer and data storage layer such as a relational database system using structured query language (SQL) or no SQL, an object store, a graph database, a flat file system or other data storage.

Computer system 805 can send messages and receive data and instructions, including program code, through the network(s), network link 865 and communication interface 860. In the Internet example, a server 855 might transmit a requested code for an application program through Internet 880, ISP 875, local network 870 and communication interface 860. The received code may be executed by processor 810 as it is received, and/or stored in storage 815, or other non-volatile storage for later execution.

The execution of instructions as described in this section may implement a process in the form of an instance of a computer program that is being executed and consisting of program code and its current activity. Depending on the operating system (OS), a process may be made up of multiple threads of execution that execute instructions concurrently. In this context, a computer program is a passive collection of instructions, while a process may be the actual execution of those instructions. Several processes may be associated with the same program; for example, opening up several instances of the same program often means more than one process is being executed. Multitasking may be implemented to allow multiple processes to share processor 810. While each processor 810 or core of the processor executes a single task at a time, computer system 805 may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

### OBJECT OF THE INVENTION

The present invention is intended to remedy all or part of the disadvantages of the prior art.

To this effect, according to a first aspect, the present invention aims at an aquatic installation monitoring system, comprising:
at least one physical/chemical sensor, configured to provide series of at least one sensed value representative of a local physical/chemical parameter, and
at least one optical sensor, and
an aquatic installation state determination means, comprising a computing device configured to receive and to process at least one series of a local physical/chemical parameter and/or at least one graphical representation to determine a value representative of an aquatic installation state.

Such provisions allow for the accurate determination of issues and risks associated with the water in the installation and/or with the installation itself.

In particular embodiments, the system object of the present invention comprises a submersible and/or floating vehicle, comprising:
at least one said physical/chemical sensor, configured to provide series of at least one sensed value representative of a local physical/chemical parameter, and/or
at least one said optical sensor, configured to provide a graphical representation of water in the aquatic installation and/or the aquatic installation.

The ability to use a mobile vehicle onboarding sensors allows for greater flexibility on the positioning of said sensors in relation to a particular physical/chemical value to be analyzed. The adequation between positioning and measurement ensures greater accuracy of the results and improved diagnostics capabilities.

In particular embodiments, at least one water physical/chemical sensor is, but is not limited to:
a pH sensor, and/or
a total alkalinity sensor, and/or
a conductivity sensor, and/or
an oxidation-reduction potential sensor, and/or
a free chlorine sensor, and/or
a total chlorine sensor, and/or
a disinfectant rate sensor, and/or
a turbidity sensor, and/or
a temperature sensor, and/or
an optical sensor, and/or
an infrared sensor, and/or
a sonar sensor, and/or
a flow sensor, and/or
a water movement sensor, and/or
a pressure sensor, and/or
a bacterial and/or algae activity sensor, and/or
a phosphate sensor, and/or
a nitrogen compounds sensor, and/or
a chloride sensor.

In particular embodiments, the optical sensor may comprise an infrared sensor.

Such embodiments allow for the optical determination of the temperature of the water in the installation and/or of the installation environment. Such temperature may be determined locally by circulating the vehicle in a variety of locations in the installation and/or by using an external sensor.

In particular embodiments, the submersible and/or floating vehicle comprises a sonar sensor configured to provide an output, the aquatic installation state determination means being configured to determine an aquatic installation state as a function of the output provided by the sonar sensor.

Such embodiments allow for the diagnostic of anomalies in a hydraulic circuit associated with the installation, such as a pump malfunction resulting in vibrations perceivable by the sonar sensor.

In particular embodiments, the system object of the present invention comprises a water physical/chemical treatment device, configured to modify a water physical/chemical parameter as a function of the aquatic installation state determined.

Such embodiments allow for the treatment of the water in an aquatic installation to correct the chemical/physical parameter so as to reach an acceptable target value or range of values.

Such embodiments allow for the treatment of the water in an aquatic installation to manage the free and/or total chlorine and/or combined chlorine rate so as to reach an acceptable target value or range of values.

Such embodiments allow for the treatment of the water in an aquatic installation to manage the disinfection rate so as to reach an acceptable target value or range of values.

In particular embodiments, the water physical/chemical sensor is configured to measure a flow intensity from an inlet in the installation, the aquatic installation state determination means being configured to determine a pump flow efficiency as a function of the flow intensity.

In particular embodiments, the aquatic installation state determination means is configured to determine a water clarity value as a function of a graphical representation provided.

In particular embodiments, the aquatic installation state determination means is configured to determine a value representative of the presence of particles in the water as a function of a graphical representation provided.

In particular embodiments, the aquatic installation state determination means is configured to determine a value representative of the presence of the nature of an impurity in the water as a function of a graphical representation provided.

Such embodiments allow for the determination of the presence of leaves, algae, mushrooms or spots (such as stain) in the installation.

In particular embodiments, the aquatic installation state determination means is configured to determine a value representative of the presence of an animal in the water as a function of a graphical representation provided.

Such embodiments allow for the detection of an intrusion in the installation.

In particular embodiments, the aquatic installation state determination means is configured to determine a value representative of a movement pattern of the animal in the water as a function of a graphical representation provided.

Such embodiments allow for the detection of a person drowning in the installation.

In particular embodiments, the system object of the present invention may comprise an aquatic total alkalinity measurement device, comprising:
a pH probe configured to measure pH at the boundary layer of a body of water,
a floating reference device in proximity of the pH probe,
a probe controller, configured to sequentially activate and deactivate, or connect and disconnect, the pH probe,
a pH measurement variation detection device, configured to detect a variation of pH measurement in a sequence of pH probe measurements, and
an aquatic total alkalinity value determination device, configured to determine an aquatic total alkalinity value of the body of water as a function of the pH measurement variation detected.

Such provisions allow for the accurate and near real-time measurement of a total alkalinity value of a body of water at an inexpensive cost and with ordinary equipment. However, the benefits of this invention result from the counter-intuitive discovery, by the inventors, which switching the pH probe on and off when the water is not flowing provides an accurate total alkalinity measurement whereas continuous pH measurement does not. This is because the successive activation/deactivation, or connection/disconnection of the pH probe results in a chemical reaction taking place in the vicinity of the pH probe. This chemical reaction results in a variation in pH measurement, said variation being dependent on the total alkalinity value of the water in the vicinity of the pH probe off, when the water is not flowing. Therefore, the present invention allows for the determination of the total alkalinity value of a body of water without using a total alkalinity measurement sensor. Such an indirect measurement significantly improves the capacity to measure total alkalinity in aquatic installations and in any other water storage and management systems.

According to a second aspect, the present invention aims at an aquatic installation monitoring method, comprises:
a step of operating:
   at least one water physical/chemical sensor, and
   at least one optical sensor.
a step of providing a sensed graphical representation of the water and/or aquatic installation, and
an aquatic installation state determination step, comprising a computing device configured to receive and to process measures of a local physical/chemical state of the water and/or graphical representations of the water to determine a value representative of an aquatic installation state and/or of its environment.

The method object of the present invention provides the same advantages as the system object of the present invention.

1. Aquatic installation monitoring system, which comprises:
at least one physical/chemical sensor, configured to provide series of at least one sensed value representative of a local physical/chemical parameter, and
at least one optical sensor, and
an aquatic installation state determination means comprising a computing device configured to receive and to process at least one series of a local physical/chemical parameter and/or at least one graphical representation to determine a value representative of an aquatic installation state.

2. System according to claim 1, which comprises a submersible and/or floating vehicle, comprising:
at least one said physical/chemical sensor, configured to provide series of at least one sensed value representative of a local physical/chemical parameter, and/or
at least one said optical sensor, configured to provide a graphical representation of water in the aquatic installation and/or the aquatic installation.

3. System according to claim 1, in which at least one water physical/chemical sensor is, but is not limited to:
a pH sensor, and/or
a total alkalinity sensor, and/or
a conductivity sensor, and/or
an oxidation-reduction potential sensor, and/or
a free chlorine sensor, and/or
a total chlorine sensor, and/or
a disinfectant rate sensor, and/or
a turbidity sensor, and/or
a temperature sensor, and/or
a flow sensor, and/or
an optical sensor, and/or
an infrared sensor, and/or
a sonar sensor, and/or
a water movement sensor, and/or
a pressure sensor, and/or
a bacterial and/or algae activity sensor, and/or
a phosphate sensor, and/or
a nitrogen compounds sensor, and/or
a chloride sensor.

4. System according to claim 1, in which the optical sensor comprises an infrared sensor.

5. System according to claim 1, in which a submersible and/or floating vehicle comprises a sonar sensor configured to provide an output, the aquatic installation state determination means being configured to determine an aquatic installation state as a function of the output provided by the sonar sensor.

6. System according to claim 1, which comprises a water physical/chemical treatment device, configured to modify a water physical/chemical parameter as a function of the aquatic installation state determined.

7. System according to claim 1, in which the water physical/chemical sensor and/or the external sensor is configured to measure a flow intensity from an inlet in the installation, the aquatic installation state determination means being configured to determine a pump flow efficiency as a function of the flow intensity.

8. System according to claim 1, in which the aquatic installation state determination means is configured to determine a water clarity value as a function of a graphical representation provided.

9. System according to claim 1, in which the aquatic installation state determination means is configured to determine a value representative of the presence of particles in the water as a function of a graphical representation provided.

10. System according to claim 1, in which the aquatic installation state determination means is configured to determine a value representative of the presence of the nature of an impurity in the water as a function of a graphical representation provided.

11. System according to claim 1, in which the aquatic installation state determination means is configured to determine a value representative of the presence of an animal in the water as a function of a graphical representation provided.

12. System according to claim 11, in which the aquatic installation state determination means is configured to determine a value representative of a movement pattern of the animal in the water as a function of a graphical representation provided.

13. System according to claim 1, which comprises an aquatic total alkalinity measurement device, comprising:
a pH probe configured to measure pH at the boundary layer of a body of water,
a floating reference device in proximity of the pH probe,
a probe controller, configured to sequentially activate and deactivate, or connect and disconnect, the pH probe,
a pH measurement variation detection device, configured to detect a variation of pH measurement in a sequence of pH probe measurements, and
an aquatic total alkalinity value determination device, configured to determine an aquatic total alkalinity value of the body of water as a function of the pH measurement variation detected.

14. Aquatic installation monitoring method, which comprises:
a step of operating:
at least one water physical/chemical sensor, and
at least one optical sensor.
a step of providing a sensed graphical representation of the water and/or aquatic installation, and
an aquatic installation state determination step, comprising a computing device configured to receive and to process measures of a local physical/chemical state of the water and/or graphical representations of the water to determine a value representative of an aquatic installation state.
